(19) United States
(12) Patent Application Publication
(10) Pub. No.: US 2023/0212560 A1
(43) Pub. Date: Jul. 6, 2023

(54) SYSTEMS, METHODS, AND MEDIA FOR DETERMINING RELATIVE QUALITY OF OLIGONUCLEOTIDE PREPARATIONS

(71) Applicant: Arc Bio, LLC, Cambridge, MA (US)

(72) Inventors: Srihari RADHAKRISHNAN, Cambridge, MA (US); Vaishnavi NAGESH, Cambridge, MA (US); Priyashree ROY, Cambridge, MA (US); Alejandro QUIROZ-ZARATE, Cambridge, MA (US)

(21) Appl. No.: 18/040,108

(22) PCT Filed: Jul. 30, 2021
(86) PCT No.: PCT/US2021/044026

§ 371 Date: Jan. 31, 2023

Related U.S. Application Data
(60) Provisional application No. 63/059,542, filed on Jul. 31, 2020.

(51) Int. Cl. C12N 15/10

(52) U.S. Cl. CPC C12N 15/1089

(57) ABSTRACT

In accordance with some embodiments, systems, methods, and media for determining relative quality of oligonucleotide preparations. In some embodiments, a system comprises a processor programmed to: (a) receive genetic sequencing results for multiple libraries with target concentrations of oligonucleotides; (b) calculate at least one prediction band; (c) repeat (a) and (b) for multiple preparations; (d) determine boundaries for a final prediction band based on the prediction bands calculated at (b) for each of the preparations; and (e) present a report indicative of quality of the libraries, including metrics indicative of the final prediction band.

### CROSS-REFERENCE TO RELATED APPLICATIONS

This application is based on, claims the benefit of, and claims priority to U.S. Provisional Application No. 63/059,542, filed Jul. 31, 2020, which is hereby incorporated herein by reference in its entirety for all purposes.

### STATEMENT REGARDING FEDERALLY SPONSORED RESEARCH

N/A

### BACKGROUND

Oligonucleotide (sometimes referred to herein as oligos) are short DNA or RNA molecules having a specific sequence of bases that can be used for a variety of purposes. For example, a group of oligos can be used in a positive control sample provided to a sequencing device (e.g., a next generation sequencing device) to determine whether the sequencing device and/or associated sequencing processes (e.g., sequence alignment) properly identifies the sequences that are known to be present in the group of oligos that were included in the positive control sample. However, using oligos for this or other purposes can be confounded if the oligos are not of sufficiently high quality. For example, quality can be affected by factors including but not limited to the presence of additional undesired species of oligos, discrepancies in relative abundances between desired oligo species, or insufficient similarity of oligo properties to the properties of the sample types for which the oligos will be used as a control.

Accordingly, new systems, methods, and media for determining relative quality of oligonucleotide preparations are desirable.

### SUMMARY

In accordance with some embodiments of the disclosed subject matter, systems, methods, and media for determining relative quality of oligonucleotide preparations are provided.

In accordance with some embodiments of the disclosed subject matter, a system for determining relative quality of oligonucleotide preparations is provided, the system comprising: at least one hardware processor that is programmed to: (a) receive genetic sequencing results for multiple libraries each associated with a target concentration of a plurality of oligonucleotides; (b) calculate at least one prediction band based on the multiple libraries; (c) repeat (a) and (b) for a plurality of preparations; (d) determine boundaries for a final prediction band based on the prediction bands calculated at (b) for each of the plurality of preparations; and (e) cause to be presented a report indicative of quality of the oligonucleotide libraries associated with the plurality of preparations, wherein the report includes at least metrics indicative of the final prediction band.

In some embodiments, the at least one hardware processor is further programmed to: subsequent to (a) and prior to (b), (i) divide the libraries into a plurality of titer bins based on target concentration, including a high titer bin and a low titer bin; and repeat (a), (i), and (b) for each of the plurality of preparations.

In some embodiments, the at least one hardware processor is further programmed to: receive genetic sequencing results for multiple new libraries each associated with a target concentration of oligonucleotides; calculate a prediction band based on the multiple new libraries; and cause the report to include at least metrics indicative of the prediction band calculated based on the multiple new libraries.

In some embodiments, the at least one hardware processor is further programmed to: divide the new libraries into the plurality of titer bins based on target concentration, including the high titer bin and the low titer bin; and calculate a prediction band based for each titer band based on the multiple new libraries; and cause the report to include at least metrics indicative of the prediction band for the high titer bin calculated based on the multiple new libraries.

In some embodiments, the at least one hardware processor is further programmed to: cause the report to include a graphical representation of the final prediction band using a first pair of axes; and cause the report to include a graphical representation of the metrics indicative of the prediction band for the high titer bin calculated based on the multiple new libraries using the same pair of axes.

In some embodiments, each prediction band includes an upper line and a lower line, wherein the upper line and the lower line are each characterized by a slope m and an intercept c.

In some embodiments, the processor is further programmed to: generate a distribution of slopes for the upper line of each prediction band corresponding to the high titer bin; determine a range of slopes for an upper boundary for the final prediction band based on the distribution of slopes for the upper line of each prediction band corresponding to the high titer bin; generate a distribution of slopes for the lower line of each prediction band corresponding to the high titer bin; determine a range of slopes for a lower upper boundary for the final prediction band based on the distribution of slopes for the lower line of each prediction band corresponding to the high titer bin; generate a distribution of intercepts for the high titer bin; determine a range of intercepts based on the distribution of intercepts for the high titer bin; and cause the report to include the range of slopes for the upper boundary, the range of slopes for the lower boundary, and the range of intercepts.

In some embodiments, the at least one hardware processor is further programmed to: cause the report to include a graphical representation of the final prediction band using a first pair of axes; and cause the report to include a graphical representation of the metrics indicative of the prediction band calculated based on the multiple new libraries using the same pair of axes.

In some embodiments, each prediction band includes an upper line and a lower line, wherein the upper line and the lower line are each characterized by a slope m and an intercept c.

In some embodiments, the processor is further programmed to: generate a distribution of slopes for the upper line of each prediction band; determine a range of slopes for an upper boundary for the final prediction band based on the distribution of slopes for the upper line of each prediction band; generate a distribution of slopes for the lower line of each prediction band; determine a range of slopes for a lower upper boundary for the final prediction band based on the distribution of slopes for the lower line of each prediction band; generate a distribution of intercepts for the high titer bin; determine a range of intercepts based on the distribution of intercepts; and cause the report to include the range of slopes for the upper boundary, the range of slopes for the lower boundary, and the range of intercepts.

In some embodiments, the processor is further programmed to: cause the report to include a graphical representation of the final prediction band based on the range of slopes for the upper boundary, the range of slopes for the lower boundary, and the range of intercepts.

In some embodiments, the genetic sequencing results for each of the multiple libraries is indicative of a number reads corresponding to each oligonucleotide of the plurality of oligonucleotides; and the processor is further programmed to: determine, for each of the libraries, a signal value indicative of the number of reads corresponding to an average of the number of reads corresponding to each oligonucleotide of the plurality of oligonucleotides;

calculate a ratio of target concentration for each pair of libraries in the multiple libraries by dividing the higher target concentration of the pair by the lower target concentration of the pair; calculate a ratio of signal values for each pair of libraries in the multiple libraries by dividing the signal value associated with the sample with the higher target concentration of the pair by the signal value associated with the sample with the lower target concentration of the pair; calculate a logarithm of each ratio of target concentration; calculate a logarithm of each ratio of signal values; and calculate the prediction band based on a plurality of points each having an x value corresponding to the logarithm of the ratio of target concentration of two libraries and a y value corresponding to the logarithm of the ratio of signal values of the two libraries.

In accordance with some embodiments of the disclosed subject matter a method for determining relative quality of oligonucleotide preparations is provided, the method comprising: (a) receiving genetic sequencing results for multiple libraries each associated with a target concentration of a plurality of oligonucleotides; (b) calculating at least one prediction band based on the multiple libraries; (c) repeating (a) and (b) for a plurality of preparations; (e) determining boundaries for a final prediction band based on the prediction bands calculated at (b) for each of the plurality of preparations; and (e) causing to be presented a report indicative of quality of the oligonucleotide libraries associated with the plurality of preparations, wherein the report includes at least metrics indicative of the final prediction band.

In accordance with some embodiments of the disclosed subject matter, a non-transitory computer readable medium containing computer executable instructions that, when executed by a processor, cause the processor to perform a method for determining relative quality of oligonucleotide preparations is provided, the method comprising: (a) receiving genetic sequencing results for multiple libraries each associated with a target concentration of a plurality of oligonucleotides; (b) calculating at least one prediction band based on the multiple libraries; (c) repeating (a) and (b) for a plurality of libraries; (d) determining boundaries for a final prediction band based on the prediction bands calculated at (b) for each the high titer bin associated with each of the plurality of libraries; and (e) causing to be presented a report indicative of quality of the oligonucleotide libraries associated with the plurality of libraries, wherein the report includes at least metrics indicative of the final prediction band.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

The patent or application file contains at least one drawing executed in color. Copies of this patent or patent application publication with color drawing(s) will be provided by the Office upon request and payment of the necessary fee.

FIG. 1 shows an example of a system for determining relative quality of oligonucleotide preparations in accordance with some embodiments of the disclosed subject matter.

FIG. 2 shows an example of hardware that can be used to implement a computing device, and a server, shown in FIG. 1 in accordance with some embodiments of the disclosed subject matter.

FIG. 3 shows an example of a process for determining relative quality of oligonucleotide preparations in accordance with some embodiments of the disclosed subject matter.

FIG. 4 shows an example of oligo libraries from a particular oligo preparation (e.g., from a particular oligo pool) grouped into bins by titer concentration in accordance with some embodiments of the disclosed subject matter.

FIG. 5A shows an example of idealized prediction bands.

FIG. 5B shows an example of oligo results generated in practice.

FIG. 6 shows examples of prediction bands for a high titer bin and a low titer bin generated from results for various preparations of oligonucleotides in accordance with some embodiments of the disclosed subject matter.

FIG. 7A shows examples of histograms of slope and intercept associated with prediction bands for a high titer bin and a low titer bin across results for various preparations of oligonucleotides in accordance with some embodiments of the disclosed subject matter.

FIG. 7B shows examples of intervals to define a final prediction band overlaid on the histograms of slope and intercept associated with prediction bands for a high titer bin and a low titer bin across results for various preparations of oligonucleotides in accordance with some embodiments of the disclosed subject matter.

FIG. 8 shows an example of prediction bands for various individual preparations and a final prediction band that can be used as a reference to determine whether quality of a new preparation(s) of oligonucleotides is acceptable in accordance with some embodiments of the disclosed subject matter.

FIG. 9 shows an example of a comparison of a final prediction band and a prediction band for a new preparation of oligonucleotides that can be used to determine whether the new preparation is acceptable in accordance with some embodiments of the disclosed subject matter.

FIG. 10 shows another example of a comparison of a final prediction band and a prediction band for a new preparation of oligonucleotides that can be used to determine whether the new preparation is acceptable in accordance with some embodiments of the disclosed subject matter.

FIG. 11 shows an example of prediction bands for various preparations of oligonucleotides plotted with each other that can be used to compare relative quality of the preparation in accordance with some embodiments of the disclosed subject matter.

FIG. 12 shows an example table of oligonucleotide libraries grouped into titer bins based on the relative concentration of oligonucleotides in accordance with some embodiments of the disclosed subject matter.

FIG. 13 shows an example of prediction bands of various subgroups of the oligonucleotides in the high titer bin described in connection with FIG. 12 and a final prediction band for each subgroup that can be used as a reference to determine relative quality of the oligonucleotide subgroups and/or relative quality of a new preparation of oligonucleotides in accordance with some embodiments of the disclosed subject matter.

FIG. 14 shows an example of plots of slope and intercept of the prediction bands for subgroup D of the ERCC oligonucleotides described in connection with FIG. 12.

FIG. 15 shows an example of prediction bands of various subgroups of the oligonucleotides in the low titer bin described in connection with FIG. 12 and a final prediction band for each subgroup that can be used as a reference to determine relative quality of the oligonucleotide subgroups and/or relative quality of a new preparation of oligonucleotides in accordance with some embodiments of the disclosed subject matter.

FIG. 16 shows an example of slopes and intercepts of prediction bands of various oligonucleotides subgroups and boxes depicting final prediction bands that can be used as a reference to determine relative quality of the oligonucleotide subgroups and/or relative quality of a new preparation of oligonucleotides in accordance with some embodiments of the disclosed subject matter.

### DETAILED DESCRIPTION

In accordance with various embodiments, mechanisms (which can, for example, include systems, methods, and media) for determining relative quality of oligonucleotide preparations are provided.

In accordance with some embodiments of the disclosed subject matter, mechanisms described herein can be used to determine metrics that can be indicative of the quality of a preparation of oligos and/or the quality of a process for sequencing a library derived from a preparation of oligos. In general, oligos can be used as normalization controls (which can sometimes be referred to as quantitative controls) that can be used to determine whether a genetic sequencing process is producing accurate and precise results. In some embodiments, a preparation of oligos can refer to a group of oligos synthesized based on a design that specifies a set of oligos based on various parameters. For example, a preparation of oligos can be a master, which can refer to a collection of oligos synthesized based on a particular design during a particular period of time. In a more particular example, a master can be X total moles of oligos based on a design specifying a set of Y different oligos at one or more target concentrations (e.g., each oligo in a design can be associated with a target molar concentration per liter, a target number of nanomoles, etc., which may be the same across all oligos or different for different sets of one or more oligos). As another example, a preparation of oligos can be a pool, which can refer to a portion of a master. In a particular example, if a particular master originally comprises 1 liter of solution, a 100 milliliter portion of the master can be referred to as a pool of oligos. As yet another example, a preparation of oligos can be a sample, which can refer to a portion of a master or pool of oligos. In a more particular example, a sample can refer to a portion of a master or pool that is to be prepared for sequencing (e.g., using one or more next generation sequencing techniques). As still another example, a preparation of oligos can be a library, which can refer to a sample or a portion of a sample that has been prepared such that it is suitable for sequencing (e.g., by ligating an adapter that the sequencing technique utilizes during sequencing to each end of the oligos). In some embodiments, multiple libraries (e.g., at different target concentrations) can be derived from a single sample by subdividing the sample and combining the portion of the sample with an amount of solvent needed to achieve a particular target concentration, where the amount of solvent needed to achieve a particular target concentration can be determined based on the target concentration of the sample.

In some embodiments, mechanisms described herein can be used to, among other things, indicate the quality of a particular oligo preparation, to compare quality between oligo preparation, to compare oligos corresponding to different experimental designs, and/or to compare oligos manufactured via different manufacturing techniques.

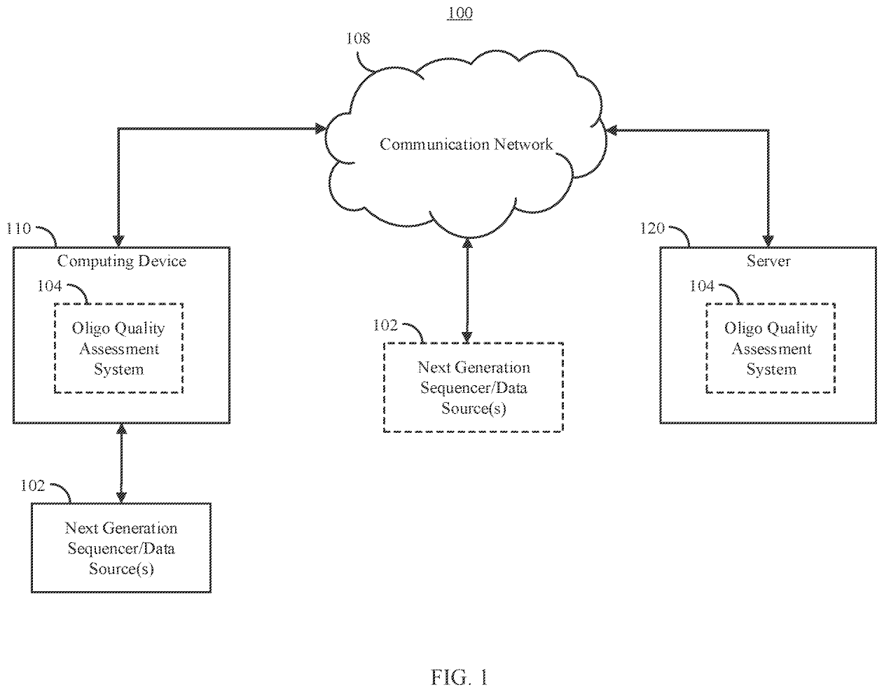

FIG. 1 shows an example 100 of a system for determining relative quality of oligonucleotide preparations in accordance with some embodiments of the disclosed subject matter. As shown in FIG. 1, a computing device 110 can receive sequencing results indicating genetic information (e.g., DNA, RNA, etc.) that is present in a library (e.g., a library prepared from a sample drawn from a master or pool including known oligonucleotides at a particular target concentration) from a data source 102 that generated and/or stores such data, and/or from an input device. In some embodiments, computing device 110 can execute at least a portion of an oligo quality assessment system 104. In some embodiments, oligo quality assessment system 104 can determine one or more quality characteristics that can be used to characterize the library or libraries and/or a preparation from which the library was derived (e.g., a sample, pool, and/or master), and which can be used to determine the relative quality of new and/or different preparations.

In some embodiments, system 100 can include an alignment system that can use any suitable alignment technique or combination of techniques, such as linear alignment techniques, and graph-based alignment techniques (e.g., as described in U.S. Patent Application Publication No. 2020/0090786, which is hereby incorporated by reference herein in its entirety) to assemble reads in results received from data source 102 into sequences (e.g., sequences corresponding to oligos in the library).

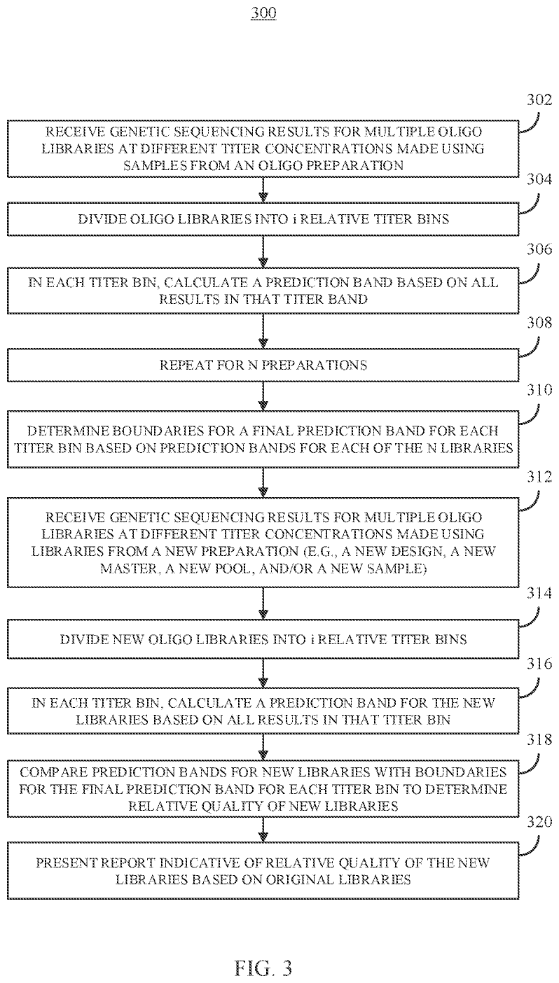

In some embodiments, oligo quality assessment system 104 can determine prediction bands based on the known target concentration of the libraries and the sequencing results received from data source 102 for the libraries. For example, oligo quality assessment system 104 can execute one or more portions of process 300 described below in connection with FIG. 3.

Additionally or alternatively, in some embodiments, computing device 110 can communicate information about genetic information (e.g., genetic sequence results generated by a next generation sequencing device, aligned reads associated with a particular library) from data source 102 to a server 120 over a communication network 108 and/or server 120 can receive genetic information from data source 102 (e.g., directly and/or using communication network 108), which can execute at least a portion of oligo quality assessment system 104. In such embodiments, server 120 can return analysis results to computing device 110 (and/or any other suitable computing device) indicative of quality of the oligo preparations.

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, a specialty device (e.g., a next generation sequencing device), etc. As described below in connection with FIG. 3, in some embodiments, computing device 110 and/or server 120 can receive genetic data (e.g., corresponding to a library or libraries including known oligonucleotides at a particular target concentration) from one or more data sources (e.g., data source 102), and can determine a final prediction band indicative of quality of the preparation from which the libraries were derived based on a signal corresponding to concentration of the oligos found in the library and the target concentration(s) for the oligos. In some embodiments, any suitable signal can be used to represent the concentration of oligos found in the results. For example, the signal can be based on a statistical transform of the number of reads that is based on a normalized ratio of reads for each oligo to total reads, which can be referred to as reads per million reads (RPM). As another example, the signal can be based on a statistical transform of the number of reads that is based on a normalized ratio of length (in bases) for each oligo to total length of all oligos, which can be referred to as reads per kilobase (RPK). As yet another example, multiple normalization bases can be used, such as normalization based on total reads and length, which can be referred to as reads per kilobase per million reads (RPKM). In some embodiments, the signal can be further normalized to the entirety of signal from a particular preparation or library, such as to the total RPM, RPK, RPKM, etc., in that preparation or library. In some embodiments, the signal value (e.g., a normalized signal value such as RPM, RPK, or RPKM) of each oligo can be used for the calculation of the prediction band. For example, RPM(oligo_i) can be the (number of reads that map to oligo_i*10^6)/(total number of mapped reads), where the total number of mapped reads is the sum of reads that mapped to any reference (e.g., a reference database of genomic sequences, control sequences, etc.).

In some embodiments, data source 102 can be any suitable source or sources of genetic data. For example, data source 102 can be a next generation sequencing device or devices that generate a large number of reads from a library. As another example, data source 102 can be a data store configured to store genetic data, which may be aligned genetic data or unaligned reads.

In some embodiments, data source 102 can be local to computing device 110. For example, data source 102 can be incorporated with computing device 110. As another example, data source 102 can be connected to computing device 110 by one or more cables, a direct wireless link, etc. Additionally or alternatively, in some embodiments, data source 102 can be located locally and/or remotely from computing device 110, and provide data to computing device 110 (and/or server 120) via a communication network (e.g., communication network 108).

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, 5G NR, etc.), a wired network, etc. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

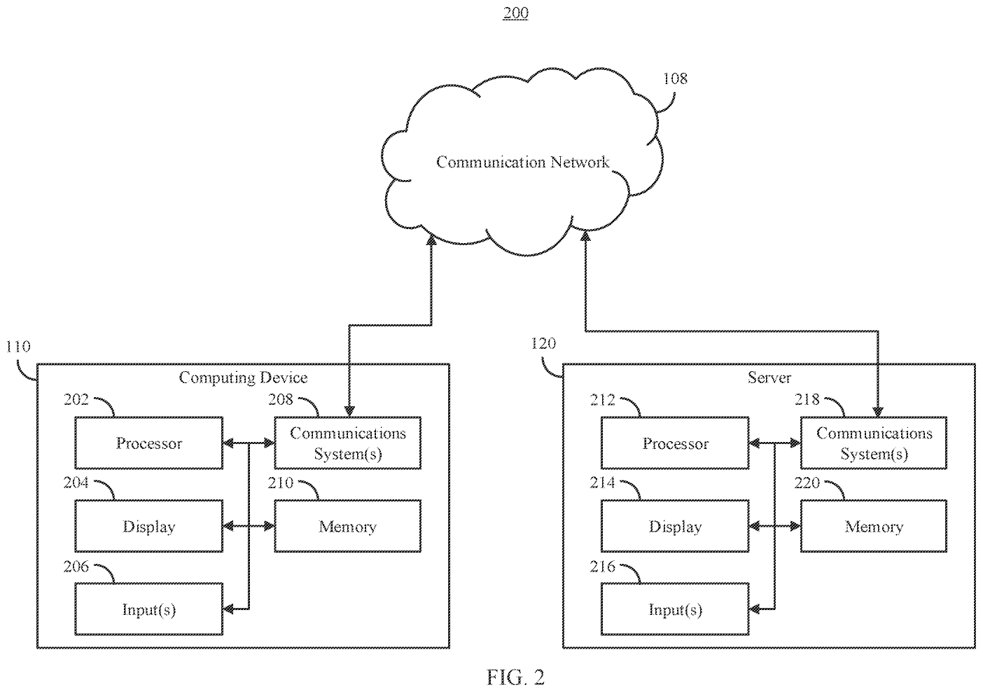

FIG. 2 shows an example 200 of hardware that can be used to implement computing device 110, and/or server 120 in accordance with some embodiments of the disclosed subject matter. As shown in FIG. 2, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), a microcontroller (MCU), an application specification integrated circuit (ASIC), a field programmable gate array (FPGA), etc. In some embodiments, display 204 can include any suitable display devices, such as a computer monitor, a touchscreen, a television, etc. In some embodiments, inputs 206 can include any suitable input devices and/or sensors that can be used to receive user input, such as a keyboard, a mouse, a touchscreen, a microphone, etc.

In some embodiments, communications systems 208 can include any suitable hardware, firmware, and/or software for communicating information over communication network 108 and/or any other suitable communication networks. For example, communications systems 208 can include one or more transceivers, one or more communication chips and/or chip sets, etc. In a more particular example, communications systems 208 can include hardware, firmware and/or software that can be used to establish a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to present content using display 204, to communicate with server 120 via communications system(s) 208, etc. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, etc. In some embodiments, memory 210 can have encoded thereon a computer program for controlling operation of computing device 110. In such embodiments, processor 202 can execute at least a portion of the computer program to present content (e.g., user interfaces, graphics, tables, reports, etc.), receive genetic data, information, and/or content from data source 102, receive information (e.g., content, genetic information, etc.) from server 120, transmit information to server 120, etc.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an MCU, an ASIC, an FPGA, etc. In some embodiments, display 214 can include any suitable display devices, such as a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 108 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, etc.

In some embodiments, memory 220 can include any suitable storage device or devices that can be used to store instructions, values, etc., that can be used, for example, by processor 212 to present content using display 214, to communicate with one or more computing devices 110, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. In such embodiments, processor 212 can execute at least a portion of the server program to transmit information and/or content (e.g., a user interface, graphs, tables, reports, etc.) to one or more computing devices 110, receive genetic data, information, and/or content from one or more computing devices 110, receive instructions from one or more devices (e.g., a personal computer, a laptop computer, a tablet computer, a smartphone, etc.), etc.

FIG. 3 shows an example of a process 300 for determining relative quality of oligonucleotide preparations in accordance with some embodiments of the disclosed subject matter. At 302, process 300 can receive genetic sequencing results for multiple oligo libraries at different target titer concentrations (e.g., each library can correspond to a test run for a particular preparation, such as a particular pool or master from which the libraries were derived and/or a particular sample if multiple libraries were derived from the same sample). In some embodiments, each oligo library can be generated from a particular preparation of oligos that include various known DNA or RNA sequences. As described above, the term “library” can refer to a plurality (e.g., collection) of oligonucleotides, e.g., a plurality of different oligonucleotides, derived from a preparation (e.g., a sample, a pool, a master, etc.). In some embodiments, a preparation from which comprises a plurality of oligonucleotides produced by fragmenting a larger nucleic acid, for example via physical (e.g., shearing), enzymatic (e.g., by nuclease), and/or chemical treatment. In some embodiments, fragments can be produced by amplification (e.g., PCR) and are thus amplicons corresponding to and/or derived from a nucleic acid (e.g., a nucleic acid to be sequenced).

In some embodiments, the oligo libraries can include the same distribution of oligos and/or different distributions of oligos. For example, the oligo libraries can all be drawn from the same preparation (e.g., sample, pool, master, etc.) and can include the same distribution of oligos. As another example, the oligo libraries can be drawn from different preparations (e.g., different samples from the same pool, different samples from the same master, different samples of different pools drawn from the same master, different samples of different masters, different samples of different pools drawn from different masters, etc.) that each include the same distribution of oligos. As yet another example, the oligo libraries can be drawn from different preparations in which at least two of the preparations include a distribution of oligos that at least partially overlaps with another preparation (e.g., there may be some oligos in common and some oligos that are different). As yet another example, the oligo libraries can be drawn from different preparations that each include a distribution of unique oligos that are not present in more than one of the preparation.

In some embodiments, process 300 can receive the genetic sequencing results in any suitable format. For example, in some embodiments, genetic data received at 302 can be formatted as results from a next generation sequencing device. In more particular example, the results can be formatted as a BCL file, which includes information received from the sequencer's sensors (e.g., regarding the luminescence that represent the biochemical signal of the reaction). In such an example, process 300 can include aligning the genetic data received at 302. In such an example, the data can be converted into another format, such as a FASTQ format, that includes both a called base and a quality score for each position of a read. As another example, the genetic data received at 302 can be received as reads that include a called base and in some cases a quality score for each position of each read. In a more particular example, the results can be formatted as a FASTQ file.

In some embodiments, process 300 can receive an indication of the target concentration associated with the genetic sequencing results from any suitable source. For example, in some embodiments, process 300 can an indication of the target concentration associated with the genetic sequencing results from an input device (e.g., an input device associated with computing device 110).

In some embodiments, process 300 can receive the results and/or can format the results as two arrays of values, an array of input titer values (e.g., an input titer array) and an array of observed RPM values (e.g., an observed RPM array). In such embodiments, the elements in the arrays can ordered by library such that input titer value at n=1 corresponds to observed RPM at n=1. As another example, process 300 can receive the results and/or can format the results as a matrix (e.g., a 2×M or M×2 matrix) in which a first row (or column) corresponds to titer values, and a second row (or column) corresponds to RPM values. As yet another example, process 300 can receive the results and/or can format the results as a matrix (e.g., a 2×M×N or M×2×N matrix, or any other suitable permutation, where M is the number of libraries derived from a preparation (e.g., sample, pool, etc.) from which the largest number of libraries were derived, and N is the number of preparations being evaluated).

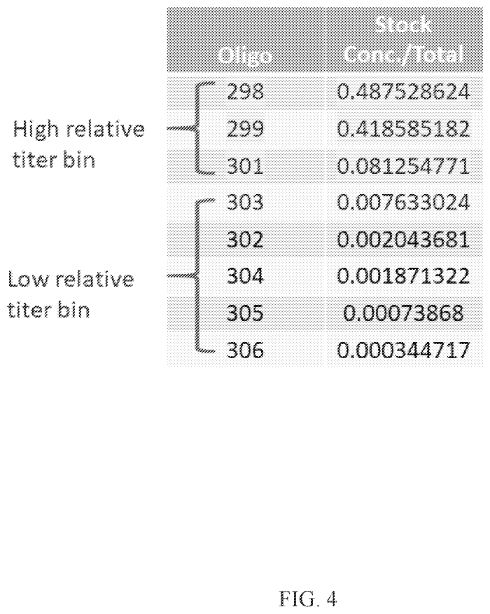

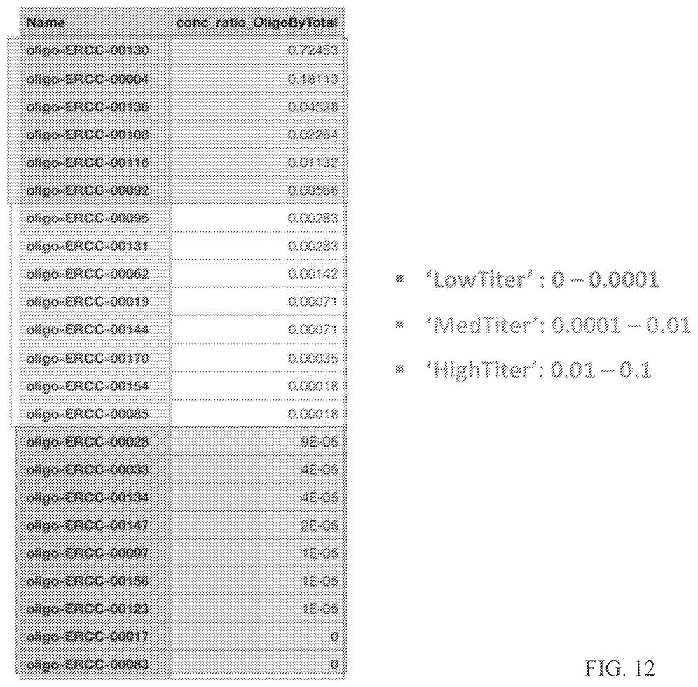

At 304, process 300 can divide the oligo libraries associated with each preparation (e.g., sample, pool, master, etc.) into i relative titer bins based on the target concentration associated with each oligo library. In some embodiments, for example as described below in connection with FIG. 4, process 300 can divide the oligo libraries associated with a preparation into a high titer bin and a low titer bin. Alternatively, in some embodiments, for example as described below in connection with FIG. 12, process 300 can divide the oligo libraries associated with a preparation into more than two bins (e.g., a low titer bin, a medium titer bin, and a high titer bin). In some embodiments, process 300 can group the oligo libraries using any suitable technique or combination of techniques. For example, process 300 can divide the oligo libraries associated with a particular preparation using any suitable technique or combination of techniques. In a more particular example, process 300 can group the libraries by identifying a median target concentration and placing the libraries above the median target concentration into a high titer bin, and placing libraries below the median target concentration into a low titer bin. In such an example, process 300 can include the library associated with the median concentration in whichever bin has a mean concentration that is closer to the median concentration. As another more particular example, process 300 can group the libraries by determining mean target concentration and placing the libraries above the mean target concentration into a high titer bin, and placing libraries below the mean target concentration into a low titer bin. As yet another more particular example, process 300 can group the libraries using one or more clustering techniques (e.g., k-means clustering, with k=2, 3, etc., corresponding to the number of desired titer bins). As still another more particular example, process 300 can group the libraries using explicit ranges received as input (e.g., ranges of concentrations associated with each titer bin can be provided by a user). In general, the titer bins selected at 304 can be used when analyzing a new preparation.

In some embodiments, process 300 can omit 304. For example, in lieu of dividing the oligo libraries associated with each preparation (e.g., sample, pool, master, etc.) into i relative titer bins, process 300 can use a single titer bin (e.g., with a range that includes all of the concentration). As another example, in lieu of dividing the oligo libraries into multiple titer bins, process 300 can utilize a single titer bin (e.g., such that i=1).

At 306, process 300 can calculate one or more prediction bands (e.g., for all libraries in the preparation, for a subset of libraries in the preparation such as for libraries that have a signal above a threshold, for each titer bin based on all results in that titer bin, etc.). In some embodiments, process 300 can remove libraries that failed. For example, process 300 can remove libraries with results having a signal below a particular threshold level (e.g., samples for which results have a value of 0). In some embodiments, process 300 can record the identity of the libraries that failed, which can be used when evaluating quality of a preparation from which the sample(s) used to derive the libraries was drawn.

In some embodiments, process 300 can calculate, for pairs of library results in (e.g., for all libraries in the preparation, for a subset of libraries in the preparation such as for libraries that have a signal above a threshold, for each titer bin, etc.), a ratio of concentrations, and a ratio of signals based on the genetic sequencing results received at 302. For example, for a titer bin that includes the libraries described below in connection with FIG. 4 (i.e., libraries 298, 299, and 301 in a high titer bin, and libraries 302-306 in a low titer bin), process 300 can calculate a ratio of concentration with the higher concentration always used as the numerator or denominator when the ratio is expressed as a fraction. In a more particular example, process 300 can always divide the larger number by the smaller number (e.g., for a<b<c, process 300 can determine three ratios:

$$\frac{c}{b} ; \frac{b}{a} ; and \frac{c}{a} ) .$$

Process 300 can generate a similar ratio for the signal (e.g., RPM) associated with each result using the same relationship between libraries that was used to determine ratios for the target concentrations (e.g., for signal values aₛ, bₛ, cₛ, process 300 can determine three ratios

$$\frac{{c}_{S}}{{b}_{S}} ; \frac{{b}_{S}}{{a}_{S}} ; and \frac{{c}_{S}}{{a}_{S}}$$

regardless of the numerical values of aₛ, bₛ, cₛ). In some embodiments, a logarithm (e.g., log based 10) can be applied to each ratio. Note that this can result in negative values for the log of a ratio of the signals, as it is possible for a library with a higher target concentration to result in a lower signal level (e.g., through one or more sources of error).

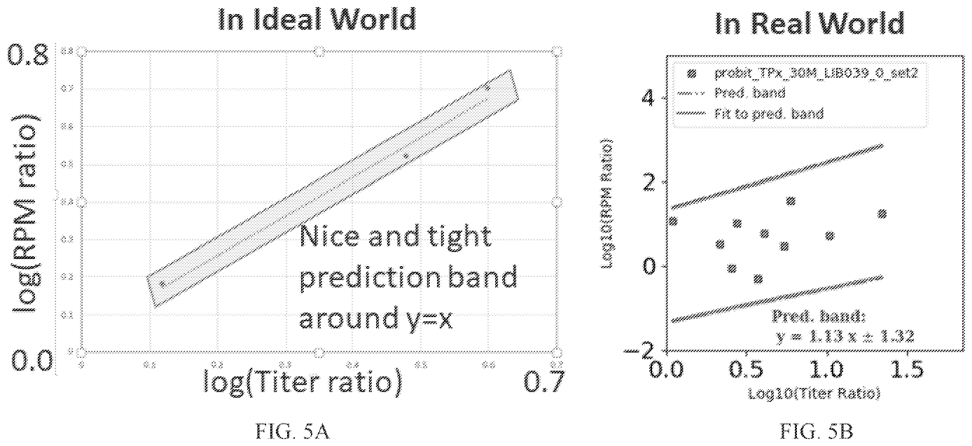

In a particular example with reference to FIG. 4, process 300 can calculate log₁₀ (concentration ratio) for each pair of library results in the high titer bin (that did not fail), to generate a set of log-ratios [0.0645; 0.777; 0.712]. Process 300 can calculate a similar set of log-ratios for the signals (e.g., RPM) associated with the genetic sequence data for each library using the same relationships. Each pair of libraries can be plotted on a log-log graph with the x value corresponding to the titer concentration ratio, and the y value corresponding to the signal (e.g., RPM) ratio. As described below in connection with FIG. 5A, because the signal should be proportional to the initial concentration, the results can be expected to closely cluster around a straight line in the log-log graph that has a slope of 1 and a y-axis intercept of 0. However, as described below in connection with FIG. 5B, the results sometimes diverge from this relationship due to various sources of error. For example, with no error the expected relationship between signal and titer concentration can be expressed as RPM₂/RPM₁=Titer₂/Titer₁. However, not all libraries generate idealized results, accordingly, a non-idealized relationship between signal and titer concentration can be expressed as RPM₂/RPM₁=Titer₂*ϵ₂(oligo₂)/Titer₁*ϵ₁(oligo1), where ϵᵢ is the efficiency of recovery, and oligo₂ titer>oligo₁ titer.

In some embodiments, process 300 can calculate a prediction band for the data corresponding to the pairwise ratios. For example, process 300 can generate a 95% prediction band for the data. A 95% prediction band can be a band into which 95% of future measurements are expected to fall within. In some embodiments, process 300 can use any suitable technique or combination of techniques to calculate a prediction band for the data. For example, process 300 can calculate a pointwise prediction band. As another example, process 300 can calculate a simultaneous prediction band (e.g., using Bonferroni's method, or Scheffe's method to account for multiple comparisons). Note that this is merely an example, and the prediction band can be any suitable prediction band (e.g., an 80% prediction band, a 90% prediction band, etc.). In some embodiments, confidence intervals can be used in addition to, or in lieu of, prediction intervals to represent the scattered distribution.

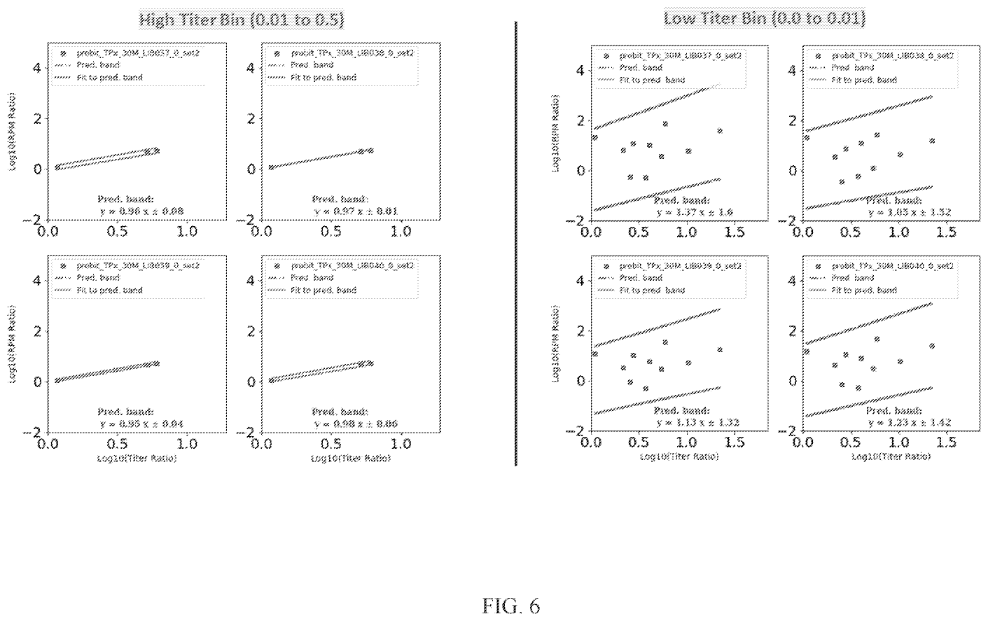

As described below in connection with FIGS. 5B and 6, the prediction band for a particular set or subset of libraries (e.g., corresponding to a particular titer bin) can include two lines which can be described using a slope m and y intercept c.

At 308, process 300 can repeat 302 to 306 for each preparation (e.g., sample, pool, master, etc.) that is being used to generate a final prediction band.

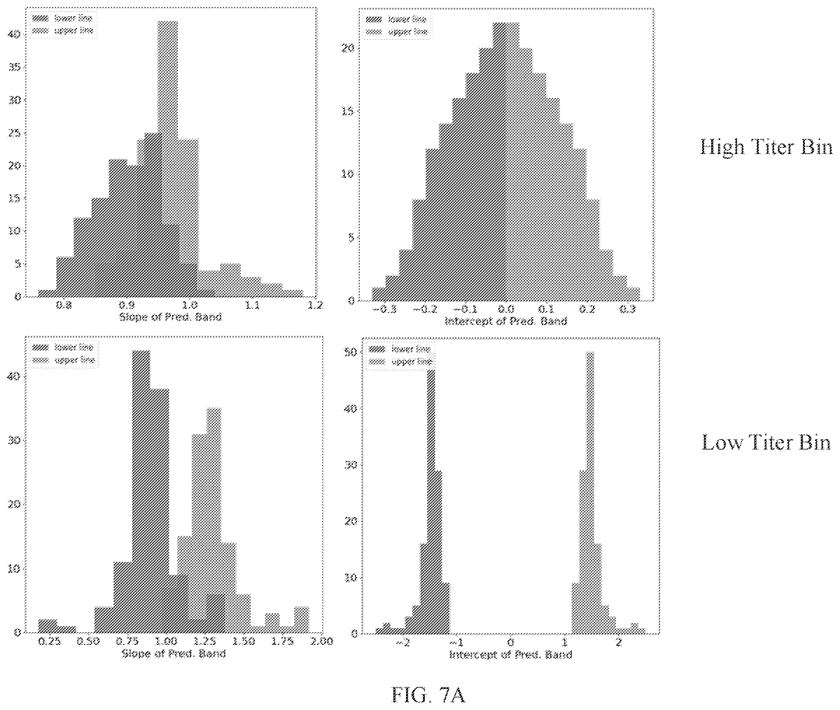

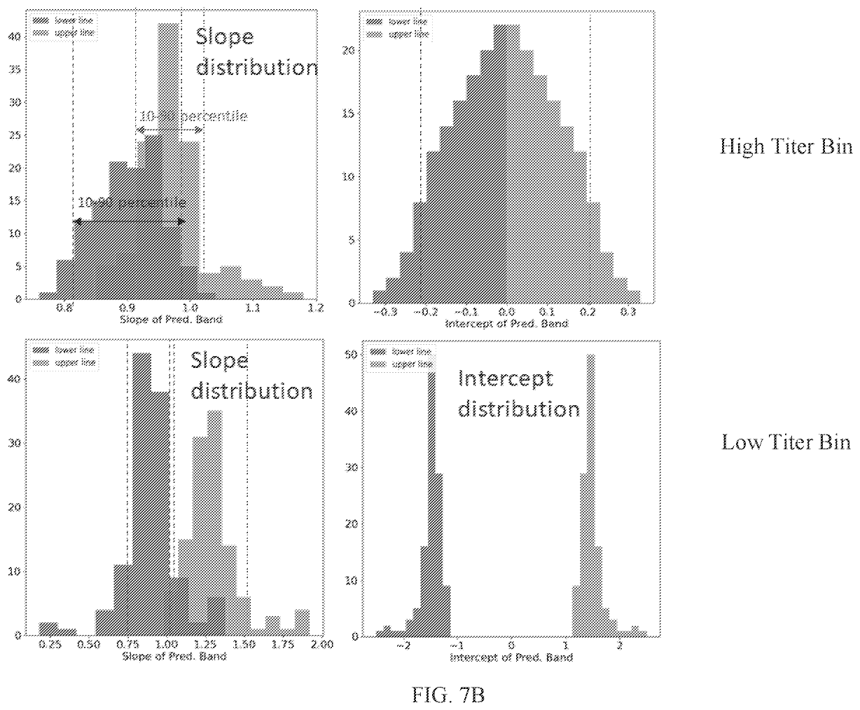

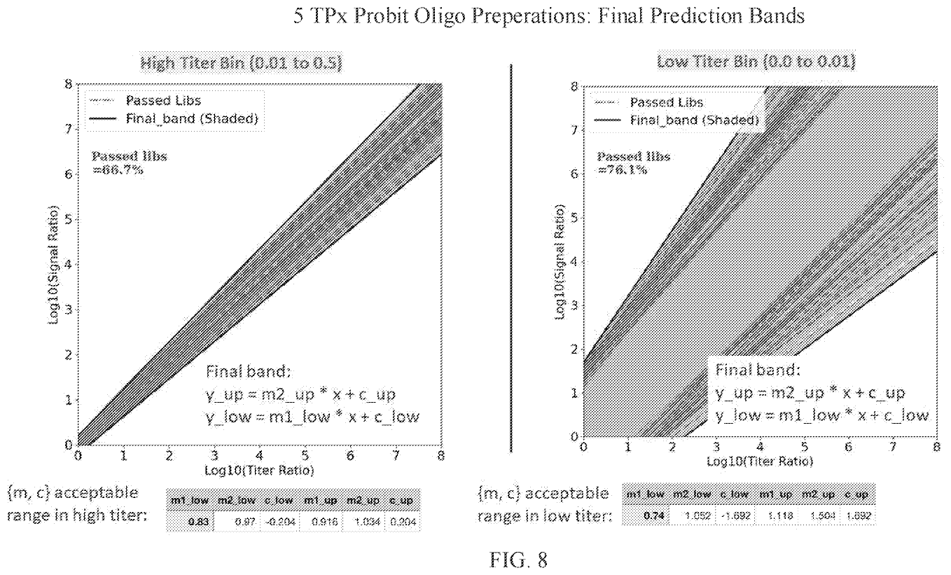

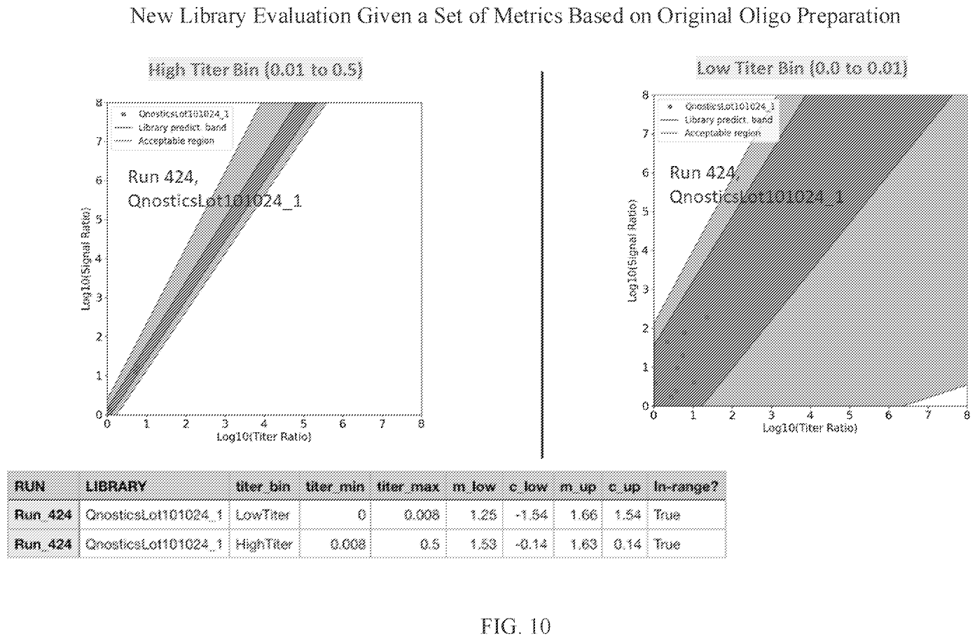

At 310, process 300 can determine boundaries for the final prediction band. For example, process 300 can determine boundaries for all libraries. As another example, process 300 can determine boundaries for all libraries that have a signal above a threshold. As yet another example, process 300 can determine boundaries for each titer bin (e.g., one set of boundaries for the high titer bin, and another set of boundaries for the low titer bin). In some embodiments, process 300 can use any suitable technique to determine the boundaries for the final prediction band(s) (e.g., based on data from all libraries derived from a particular preparation). For example, as described below in connection with FIG. 7B, process 300 can determine a range of slopes that encompass a particular percentile (e.g., 80%, 90%, 95%, etc.) of the slopes of prediction bands across all libraries in a titer bin regardless of which preparation the library was derived from (e.g., process 300 can aggregate the prediction bands across all preparations being evaluated). In a more particular example, separate values can be calculated for the upper line and lower line of the prediction bands. As another example, process 300 can determine a range of y intercepts that encompass a particular percentage of the intercepts of prediction bands across all libraries in the preparation and/or titer bin regardless of which preparation the library was derived from (e.g., process 300 can aggregate the prediction bands across all preparations being evaluated). In some embodiments, these ranges can represent metrics that can be used to evaluate the relative quality of a preparation in comparison with quality of the preparation(s) from which the libraries used to generate the final boundaries were derived (e.g., as shown in FIG. 9-10).

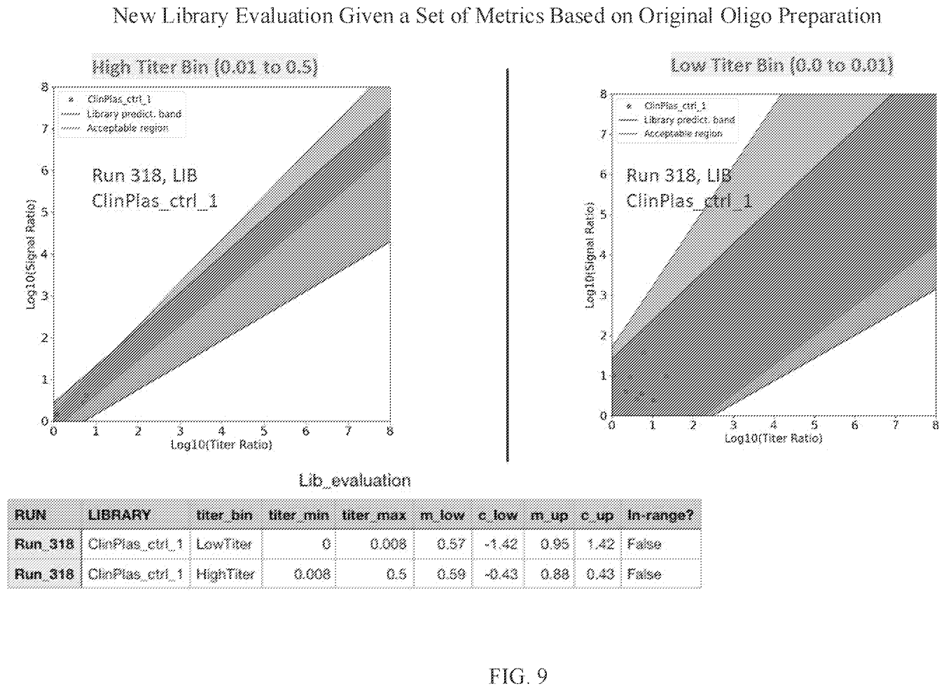

As described below in connection with FIG. 8, the final boundaries can be represented graphically as two thick black lines enclosing a shaded ‘acceptable’ region. The boundaries are determined by using the lowest slope in the selected range of slopes for the lower line and the lowest y-intercept in the selected range of intercepts for the lower line to draw lower boundary, and the highest slope in the selected range of slopes for the upper line and the highest y-intercept in the selected range of intercepts for the upper line to draw an upper boundary. In some embodiments, the final boundaries can be used to evaluate the relative quality of a library or libraries derived from a new preparation in comparison with quality of the libraries used to generate the final boundaries that characterize the corresponding oligo preparation, as shown in FIGS. 9-10. The blue shaded region shows the prediction band of the sample. If it falls within the orange-shaded final boundary (e.g. in FIG. 10), the sample is regarded as a meeting the expectations for the given oligo preparation.

At 312, process 300 can receive genetic sequencing results for multiple oligo libraries at different target titer concentrations that are drawn from a new preparation (e.g., a new master based on a new design, a new master based on the same design, a new pool prepared from the same master, etc.). In some embodiments, process 300 can receive genetic sequencing results using any suitable technique or combination of techniques, such as techniques described above in connection with 302.

At 314, process 300 can divide the new oligo libraries into i relative titer bins (e.g., one or more titer bins). In some embodiments, process 300 can use the titer concentration ranges used to divide the libraries at 304 to divide the new samples. At 316, process 300 can calculate a prediction band for each titer bin based on all results from the new libraries that are included in that titer bin. In some embodiments, process 300 can use any suitable technique or combination of techniques to calculate a prediction band, such as techniques described above in connection with 306. As described above, in some embodiments, process 300 can omit 314. For example, in lieu of dividing the oligo libraries associated with each preparation (e.g., sample, pool, master, etc.) into i relative titer bins, process 300 can use a single titer bin (e.g., with a range that includes all of the concentration). As another example, in lieu of dividing the oligo libraries into multiple titer bins, process 300 can utilize a single titer bin (e.g., such that i=1).

At 318, process 300 can generate a comparison of the prediction bands for the new libraries with the final boundaries of the prediction band (e.g., for each titer bin). In some embodiments, the comparison can be used to evaluate the quality of the new preparation(s) from which the new libraries were derived with respect to the quality of the preparation(s) used to generate the final prediction band boundaries at 310.

At 320, process 300 can present a report that is indicative of the relative quality of the new preparation(s) based on the quality of the original samples (e.g., the original samples used to generate the final prediction band at 320).

In some embodiments, the report can include any suitable information and/or graphics. For example, the report can include graphical information shown in, and described below in connection with, one or more of FIGS. 8-11.

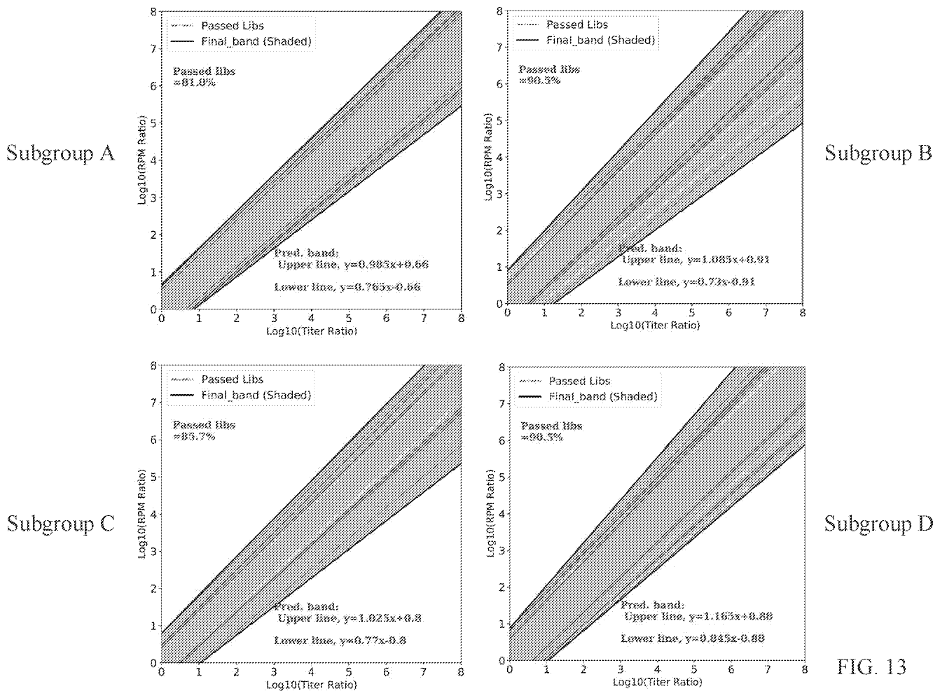

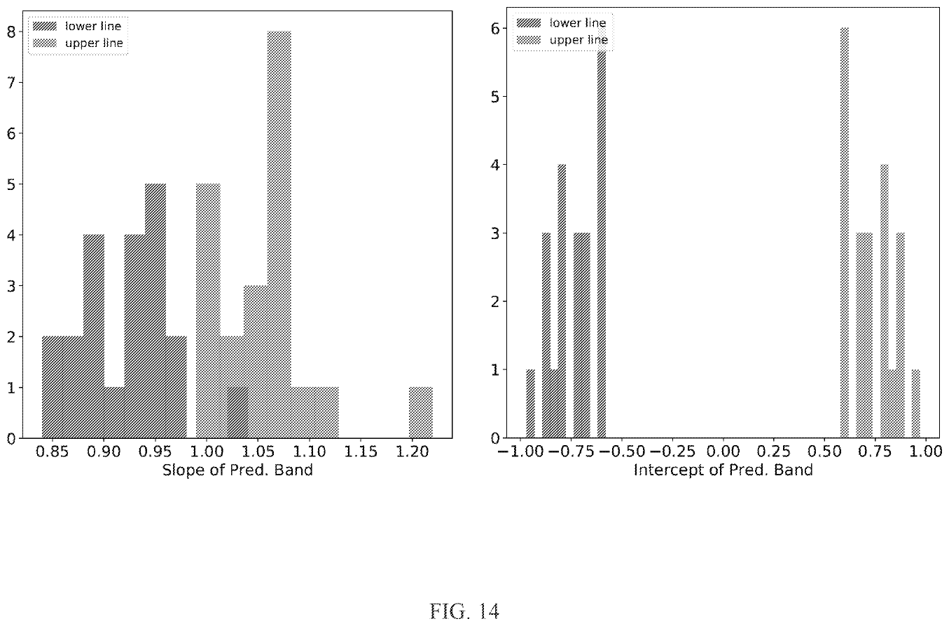

In some embodiments, 312 to 318 can be omitted, and the report can include information that is indicative of the original preparation(s) and/or that includes comparisons of various subgroups from the original libraries derived from the original preparation. For example, the report can include graphical information shown in, and described below in connection with, one or more of FIGS. 13-15.

FIG. 4 shows an example of oligo libraries from a particular oligo preparation (e.g., from a particular oligo pool) grouped into bins by titer concentration in accordance with some embodiments of the disclosed subject matter. As shown in FIG. 4, the samples are each associated with a target concentration. Concentrations for each library in FIG. 4 are expressed as a ratio of the target concentration of all oligos in that library to the concentration of all oligos in the preparation (e.g., sample, pool, master, etc.) from which the library was derived. For example, if a library is derived by diluting a sample to half the concentration of the preparation from was derived, the target concentration for the library can be expressed as 0.5 regardless of what the precise target concentration of oligos is in the preparation and the library. The samples have been divided into a high relative titer bin corresponding to relative concentrations of 0.01 to 1, and a low relative titer bin corresponding to relative concentrations of 0 to 0.01. As described above in connection with 304 of FIG. 3, any suitable technique can be used to determine ranges for two or more titer bins used to analyze the relative quality of different oligo preparations. Note that this is an example, and as described above, process 300 can omit dividing the libraries into multiple titer bins (e.g., oligo libraries 298 through 306 can be included in a single bin).

FIG. 5A shows an example of idealized prediction bands, and FIG. 5B shows an example of oligo results generated in practice. As shown in FIG. 5A, the relationship between titer concentration and a signal based on sequencing results can be expected to be linear as at 0 concentration there should be 0 signal, and as the concentration increases the signal should increase proportionally (e.g., for signals based on linear transforms).

As shown in FIG. 5B, however, results generated in the real world do not always align closely with a straight line that intercepts at y=0. This can be especially problematic for small relative concentrations, as an error in the process of generating results may not scale with concentration, and thus may have a larger impact on the final signal value for a library with a low target concentration than it would for a library with a relatively high target concentration.

FIG. 6 shows examples of prediction bands for a high titer bin and a low titer bin generated from results for various libraries of oligonucleotides in accordance with some embodiments of the disclosed subject matter. In FIG. 6 prediction bands for a high titer bin (e.g., for relative concentrations of 0.01 to 0.5) for four different libraries are shown on the left, and bands for a low titer bin (e.g., for relative concentrations of 0.0 to 0.01) for the same four libraries are shown on the right. As shown in FIG. 6, the prediction bands for the high titer bin are relatively tight (e.g., the upper and lower lines are relatively close together), and intercept the y-axis relatively close to 0 (e.g., within 0.1). By contrast, the prediction bands for the low titer bin are relatively far apart, and intercept the y-axis much father from 0 (e.g., at least 1.3 from 0). In some embodiments, techniques for generating prediction bands can produce a set of discrete values that represent the prediction band, rather than producing a pair of lines that represent the prediction band. For example, a prediction band can be represented by two arrays (or a single matrix) of values, one array of values can include y-values for the lower end of the band at various points along the x axis (i.e., at various x-values), and a second array of values can include y-values for the upper end of the band at that same points along the x axis. In some embodiments, the slope and y-intercept for each boundary of the prediction band can be determined by performing a linear fit to each array, and using the resulting line to estimate the slope and y-intercept for prediction band.

As shown in FIG. 6, each prediction band can be defined by a pair of lines each having a slope m and a y-intercept c, such that a pair can be represented by the relationship y=mx+c. Note that in practice the slopes of the upper and lower lines may be different, and so may the y-intercepts. In general, a slope that is approximately 1 can indicate that the various titer concentrations within a bin are recovered at about the same rate. A slope greater than 1 can indicate that lower titer concentrations are recovered at a lower rate than higher titer concentrations, while a slope less than 1 can indicate that higher titer concentrations are recovered at a lower rate than lower titer concentrations. As shown in FIG. 6, the slope of prediction bands in the high titer bins are all relatively close to 1, while the slopes in the lower titer bins are all higher than 1 by varying amounts. The y-intercept is a constant that can represent the difficulty of determining an accurate signal for given titer ratios. For example, for c close to 0, the band is relatively tight, and it is relatively easy to determine RPM for a given titer concentration. A larger c, by contrast, can indicate that there is more variation, making it more difficult to determine RPM for a given titer concentration. It is not clear what factors can affect c, however a potential source of a large c may be that the actual concentration of oligos in the various libraries does not correspond to the target concentration, or that the sequences in the library have shorter than expected length.

FIG. 7A and 7B shows examples of histograms of slope and intercept associated with prediction bands for a high titer bin and a low titer bin across results for various preparations of oligonucleotides, and intervals that can be used to determine a final prediction band, respectively, in accordance with some embodiments of the disclosed subject matter. In some embodiments, the slope and intercept of each line of each prediction band can define a distribution of values. As shown in FIG. 7A, the distribution of slopes for the upper and lower lines for the high titer bin are distributed close to 1, and the y-intercept is distributed around 0. However, the slopes for the low titer bin are not distributed as closely to 0, and the y-intercepts are relatively far from 0.

As shown in FIG. 7B, an interval can be determined for each slope distribution (e.g., one for the high titer bin lower line distribution, one for the high titer bin upper line distribution, one for the low titer bin lower line distribution, etc.), and an interval can be determined for the y-intercept distribution for each titer bin. Note that the ranges for slopes in FIG. 7B are from the 10^{th} percentile to the 90^{th} percentile, but this is merely an example and the range can include any suitable portion of the distribution. For example, the range included in the final prediction band metric can be 2.5-97.5 percentile. In some embodiments, the range selected for the y-intercept can include 90 percent of y-intercept values.

FIG. 8 shows an example of prediction bands for various individual preparations and a final prediction band that can be used as a reference to determine whether quality of a new preparation(s) of oligonucleotides is acceptable in accordance with some embodiments of the disclosed subject matter. As shown in FIG. 8, a final prediction band for an oligo preparation(s) represented by a group of libraries derived from a preparation of oligos based on a design that includes oligos corresponding to a transplant viral panel (TPx) for which a probit analysis was performed to establish linearity of the preparation. Libraries derived from such a preparation can be referred to as probit TPx libraries. In FIG. 8, a final prediction band corresponding to an acceptable range in a high titer bin and a low titer bin can be presented (e.g., as a table), and the prediction bands for each library that fell within the final bands can be presented in graphical form. Note than in the results in FIG. 9, 66.7% (about ⅔) of the libraries generated prediction bands that fall within the final prediction band for the high titer bin, while 76.1% of the libraries generated prediction bands that fall within the final prediction band for the low titer bin. Note that the “passed” libraries were libraries for which both the upper and lower boundary of the prediction band fell within the final bands.

FIG. 9 shows an example of a comparison of a final prediction band and a prediction band for a new preparation of oligonucleotides that can be used to determine whether the new preparation is acceptable in accordance with some embodiments of the disclosed subject matter. As shown in FIG. 9, a final prediction band for an oligo preparation(s) represented by a group of libraries is presented with a prediction band for a particular set of libraries derived from the new preparation (i.e. “Library predict.band” in blue corresponds to results from library (LIB) ClinPlas_ctrl_1). In FIG. 9, the prediction bands for the new libraries does not completely overlap with the final prediction bands generated from the original data in either the low titer bin or the high titer bin. This can indicate that the new libraries are lower quality, as the prediction bands are indicative of more variable results.

FIG. 10 shows another example of a comparison of a final prediction band and a prediction band for a new preparation of oligonucleotides that can be used to determine whether the new preparation is acceptable in accordance with some embodiments of the disclosed subject matter. As shown in FIG. 10, a final prediction band for an oligo preparation(s) represented by a group of libraries is presented with a prediction band for a particular set of libraries derived from the new preparation(s) (i.e. “Library predict.band” in blue corresponds to results from library QnosticsLot101024_1). In FIG. 10, the prediction bands for the new libraries is completely within the final prediction bands generated from the original data in both the low titer bin or the high titer bin. This can indicate that the new preparation(s) are higher quality, as the prediction bands are indicative of more consistent results.

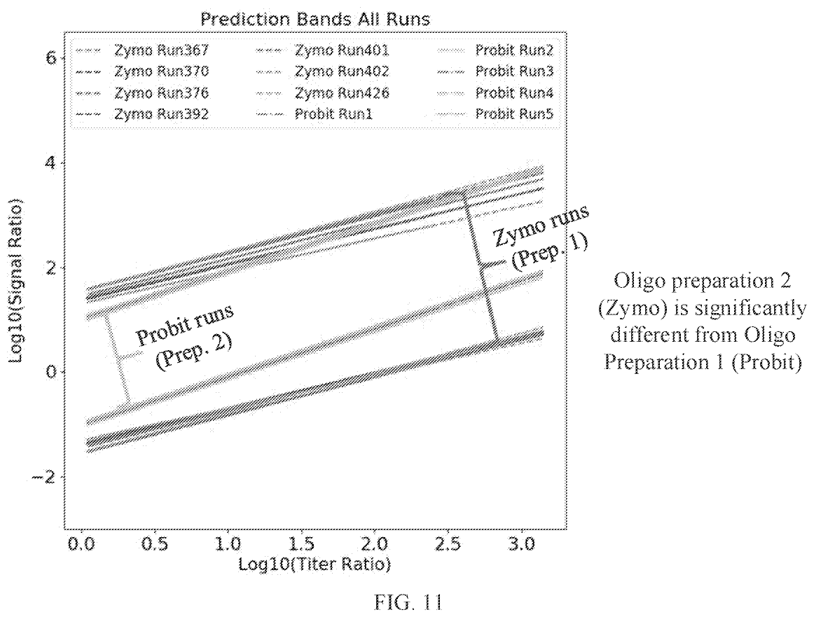

FIG. 11 shows an example of prediction bands for various preparations of oligonucleotides plotted with each other that can be used to compare relative quality of the preparations in accordance with some embodiments of the disclosed subject matter. As shown in FIG. 11, library level prediction bands for two different preparations prepared based on different designs are presented on the same graph. In FIG. 11, the prediction bands for the two different preparations is significantly different, with the probit TPx prediction bands being more consistent than the Zymo prediction bands, and covering a smaller area of the graph (e.g., indicating that the first preparation is expected to generate more consistent and more accurate results across titer concentrations). Note that the ‘Zymo’ libraries were derived from a preparation of oligos based on a design that implements a microbial community standard developed by Zymo Research Corporation, which includes oligos corresponding to 8 bacteria and 2 fungi.

FIG. 12 shows an example table of 23 oligonucleotide libraries grouped into titer bins based on the relative concentration of oligonucleotides in accordance with some embodiments of the disclosed subject matter. All of the 23 libraries in FIG. 12 correspond to one subgroup of 92 oligos specified by the External RNA Controls Consortium (ERCC) hosted by the National Institutes of Standards and Technology (NIST). The 92 RNA oligos were divided into 4 subgroups (referred to herein as subgroups A to D) that each included 23 ERCC oligos, with each subgroup having the same relative concentration distribution. The results described below in connection with FIGS. 13-15 were generated from 21 different preparations that each included all 92 ERCC-RNA oligos. As shown in FIG. 12, the libraries were divided into three titer bins, a high titer bin, a medium titer bin, and a low titer bin.

FIG. 13 shows an example of prediction bands of various subgroups of the oligonucleotides in the high titer bin described in connection with FIG. 12 and a final prediction band for each subgroup that can be used as a reference to determine relative quality of the four oligo subgroups and/or relative quality of a new preparation of oligonucleotides in accordance with some embodiments of the disclosed subject matter. As shown in FIG. 13, the final bands for each subgroup across preparations was relatively consistent in the high titer bin.

FIG. 14 shows an example of plots of slope and intercept of the prediction bands for subgroup D of the ERCC oligonucleotides described in connection with FIG. 12.

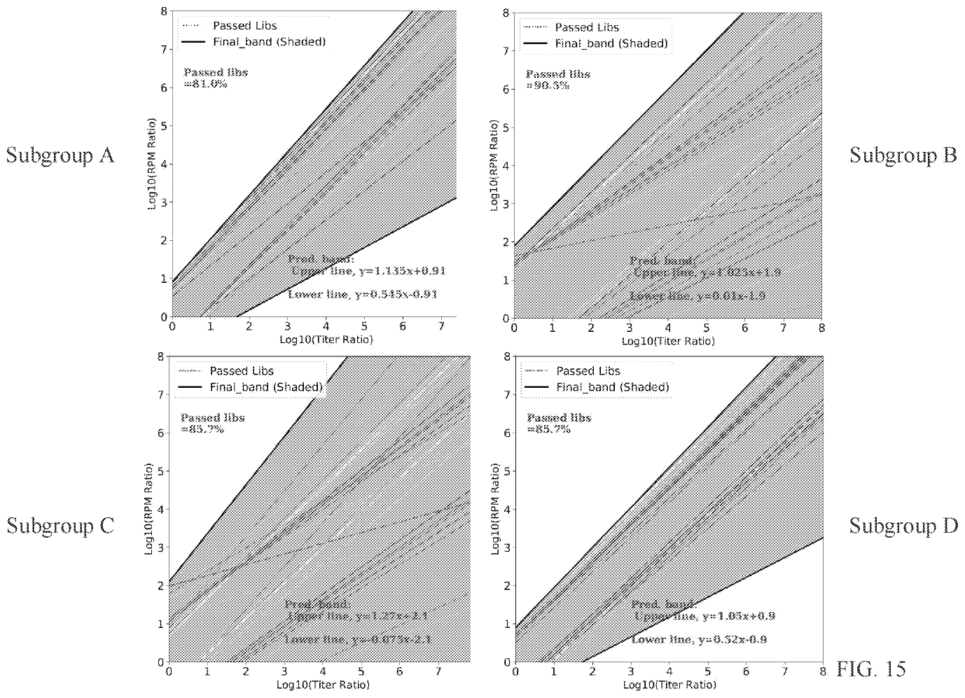

FIG. 15 shows an example of prediction bands of various subgroups of the oligonucleotides in the medium titer bin described in connection with FIG. 12 and a final prediction band for each subgroup that can be used as a reference to determine relative quality of the four oligo subgroups and/or relative quality of a new preparation of oligonucleotides in accordance with some embodiments of the disclosed subject matter. As shown in FIG. 15, the prediction bands for each subgroup across preparations was much less consistent in the medium titer bin, with subgroups B and C having much worse acceptance intervals than subgroups A and D. This may indicate that the oligos in subgroups B and C were of relatively lower quality. This is consistent with the experience of attempting to manufacture the oligos, as the oligos in subgroup C were difficult to manufacture. Accordingly, the wide acceptance intervals can be indicative of problems during manufacture (and/or other sources of error).

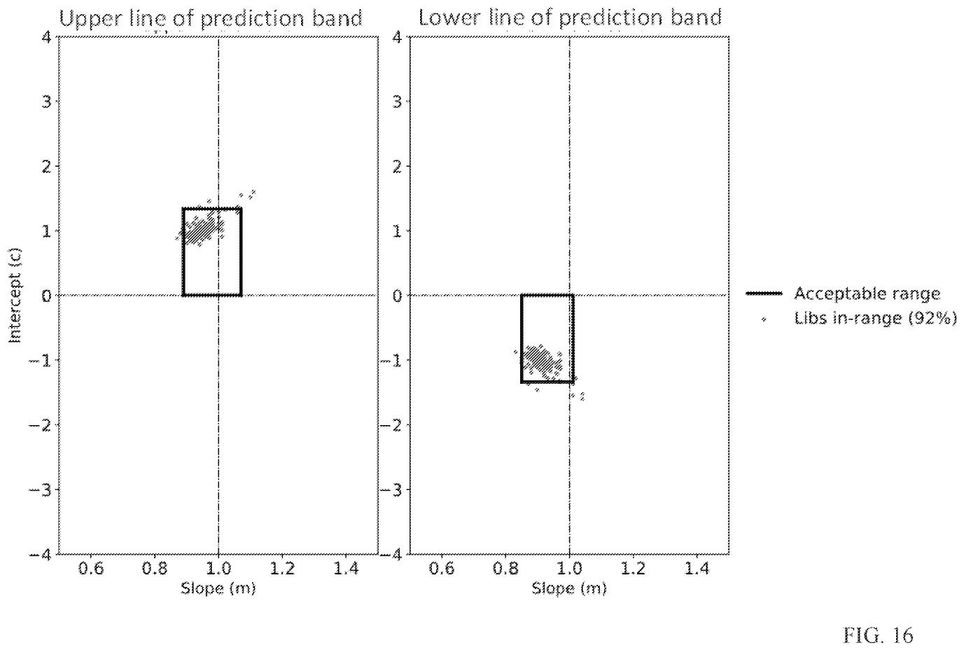

FIG. 16 shows an example of slopes and intercepts of prediction bands of various oligonucleotides subgroups and boxes depicting final prediction bands that can be used as a reference to determine relative quality of the oligonucleotide subgroups and/or relative quality of a new preparation of oligonucleotides in accordance with some embodiments of the disclosed subject matter. As shown in FIG. 16, the slope and intercept of prediction bands can be plotted for visualization, and a box can be drawn that defines a range of acceptable slope and intercept combinations. Such a visualization, or a portion of the visualization, can be presented (e.g., in connection with a report presented at 320 by process 300) to facilitate analysis by a user. For example, when a new preparation of oligonucleotides is analyzed using process 300, the slope and intercept of the upper and lower prediction bands can be plotted on a graph with a range of acceptable prediction bands plotted as a box (e.g., as shown in FIG. 16). A user can visually confirm whether the prediction bands for the new preparation fall within the box representing the acceptable range.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above described steps of the processes of FIGS. 3 to 5 can be executed or performed in any order or sequence not limited to the order and sequence shown and described in the figures. Also, some of the above steps of the processes of FIGS. 3 to 5 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. A system for determining relative quality of oligonucleotide preparations, the system comprising:
at least one hardware processor that is programmed to:
(a) receive genetic sequencing results for multiple libraries each associated with a target concentration of a plurality of oligonucleotides; (b) calculate at least one prediction band based on the multiple libraries;
(c) repeat (a) and (b) for a plurality of preparations;
(d) determine boundaries for a final prediction band based on the prediction bands calculated at (b) for each of the plurality of preparations; and
(e) cause to be presented a report indicative of quality of the oligonucleotide libraries associated with the plurality of preparations, wherein the report includes at least metrics indicative of the final prediction band.

2. The system of claim 1, wherein the at least one hardware processor is further programmed to:
subsequent to (a) and prior to (b), (i) divide the libraries into a plurality of titer bins based on target concentration, including a high titer bin and a low titer bin; and
repeat (a), (i), and (b) for each of the plurality of preparations.

3. The system of claim 1, wherein the at least one hardware processor is further programmed to:
receive genetic sequencing results for multiple new libraries each associated with a target concentration of oligonucleotides;
calculate a prediction band based on the multiple new libraries; and
cause the report to include at least metrics indicative of the prediction band calculated based on the multiple new libraries.

4. The system of claim 3, wherein the at least one hardware processor is further programmed to:
divide the new libraries into the plurality of titer bins based on target concentration, including the high titer bin and the low titer bin; and
calculate a prediction band based for each titer band based on the multiple new libraries; and
cause the report to include at least metrics indicative of the prediction band for the high titer bin calculated based on the multiple new libraries.

5. The system of claim 4, wherein the at least one hardware processor is further programmed to:
cause the report to include a graphical representation of the final prediction band using a first pair of axes; and
cause the report to include a graphical representation of the metrics indicative of the prediction band for the high titer bin calculated based on the multiple new libraries using the same pair of axes.

6. The system of claim 4, wherein each prediction band includes an upper line and a lower line, wherein the upper line and the lower line are each characterized by a slope m and an intercept c.

7. The system of claim 6, wherein the processor is further programmed to:
generate a distribution of slopes for the upper line of each prediction band corresponding to the high titer bin;
determine a range of slopes for an upper boundary for the final prediction band based on the distribution of slopes for the upper line of each prediction band corresponding to the high titer bin;
generate a distribution of slopes for the lower line of each prediction band corresponding to the high titer bin;
determine a range of slopes for a lower upper boundary for the final prediction band based on the distribution of slopes for the lower line of each prediction band corresponding to the high titer bin;
generate a distribution of intercepts for the high titer bin;
determine a range of intercepts based on the distribution of intercepts for the high titer bin; and
cause the report to include the range of slopes for the upper boundary, the range of slopes for the lower boundary, and the range of intercepts.

8. The system of claim 1, wherein the at least one hardware processor is further programmed to:
cause the report to include a graphical representation of the final prediction band using a first pair of axes; and
cause the report to include a graphical representation of the metrics indicative of the prediction band calculated based on the multiple new libraries using the same pair of axes.

9. The system of claim 1, wherein each prediction band includes an upper line and a lower line, wherein the upper line and the lower line are each characterized by a slope m and an intercept c.

10. The system of claim 9, wherein the processor is further programmed to:
generate a distribution of slopes for the upper line of each prediction band;
determine a range of slopes for an upper boundary for the final prediction band based on the distribution of slopes for the upper line of each prediction band;
generate a distribution of slopes for the lower line of each prediction band;
determine a range of slopes for a lower upper boundary for the final prediction band based on the distribution of slopes for the lower line of each prediction band;
generate a distribution of intercepts;
determine a range of intercepts based on the distribution of intercepts; and
cause the report to include the range of slopes for the upper boundary, the range of slopes for the lower boundary, and the range of intercepts.

11. The system of claim 10, wherein the processor is further programmed to:
cause the report to include a graphical representation of the final prediction band based on the range of slopes for the upper boundary, the range of slopes for the lower boundary, and the range of intercepts.

12. The system of claim 1, wherein the genetic sequencing results for each of the multiple libraries is indicative of a number reads corresponding to each oligonucleotide of the plurality of oligonucleotides; and
wherein the processor is further programmed to:
determine, for each of the libraries, a signal value indicative of the number of reads corresponding to an average of the number of reads corresponding to each oligonucleotide of the plurality of oligonucleotides;
calculate a ratio of target concentration for each pair of libraries in the multiple libraries by dividing the higher target concentration of the pair by the lower target concentration of the pair;
calculate a ratio of signal values for each pair of libraries in the multiple libraries by dividing the signal value associated with the library with the higher target concentration of the pair by the signal value associated with the library with the lower target concentration of the pair;
calculate a logarithm of each ratio of target concentration;
calculate a logarithm of each ratio of signal values; and
calculate the prediction band based on a plurality of points each having an x value corresponding to the logarithm of the ratio of target concentration of two libraries and a y value corresponding to the logarithm of the ratio of signal values of the two libraries.

13. A method for determining relative quality of oligonucleotide preparations, the method comprising:
(a) receiving genetic sequencing results for multiple libraries each associated with a target concentration of a plurality of oligonucleotides;
(b) calculating at least one prediction band based on the multiple libraries;
(c) repeating (a) and (b) for a plurality of preparations;
(d) determining boundaries for a final prediction band based on the prediction bands calculated at (b) for each of the plurality of preparations; and
(e) causing to be presented a report indicative of quality of the oligonucleotide libraries associated with the plurality of preparations, wherein the report includes at least metrics indicative of the final prediction band.

14. The method of claim 13, further comprising:
subsequent to (a) and prior to (b), (i) dividing the libraries into a plurality of titer bins based on target concentration, including a high titer bin and a low titer bin; and
repeating (a), (i), and (b) for each of the plurality of preparations.

15. The method of claim 13, further comprising:
receiving genetic sequencing results for multiple new libraries each associated with a target concentration of oligonucleotides;
calculating a prediction band based on the multiple new libraries; and
causing the report to include at least metrics indicative of the prediction band calculated based on the multiple new libraries.

16. The method of claim 15, further comprising:
dividing the new libraries into the plurality of titer bins based on target concentration, including the high titer bin and the low titer bin; and
calculating a prediction band based for each titer band based on the multiple new libraries; and
causing the report to include at least metrics indicative of the prediction band for the high titer bin calculated based on the multiple new libraries.

17. The method of claim 16, further comprising:
causing the report to include a graphical representation of the final prediction band using a first pair of axes; and
causing the report to include a graphical representation of the metrics indicative of the prediction band for the high titer bin calculated based on the multiple new libraries using the same pair of axes.

18. The method of claim 17, wherein each prediction band includes an upper line and a lower line, wherein the upper line and the lower line are each characterized by a slope m and an intercept c.

19. The method of claim 18, further comprising:
generating a distribution of slopes for the upper line of each prediction band corresponding to the high titer bin;
determining a range of slopes for an upper boundary for the final prediction band based on the distribution of slopes for the upper line of each prediction band corresponding to the high titer bin;
generating a distribution of slopes for the lower line of each prediction band corresponding to the high titer bin;
determining a range of slopes for a lower upper boundary for the final prediction band based on the distribution of slopes for the lower line of each prediction band corresponding to the high titer bin;
generating a distribution of intercepts for the high titer bin;
determining a range of intercepts based on the distribution of intercepts for the high titer bin; and
causing the report to include the range of slopes for the upper boundary, the range of slopes for the lower boundary, and the range of intercepts.

20. The method of claim 13, further comprising:
causing the report to include a graphical representation of the final prediction band using a first pair of axes; and
causing the report to include a graphical representation of the metrics indicative of the prediction band calculated based on the multiple new libraries using the same pair of axes.

21. The method of claim 13, wherein each prediction band includes an upper line and a lower line, wherein the upper line and the lower line are each characterized by a slope m and an intercept c.

22. The method of claim 21, further comprising:
generating a distribution of slopes for the upper line of each prediction band;
determining a range of slopes for an upper boundary for the final prediction band based on the distribution of slopes for the upper line of each prediction band;
generating a distribution of slopes for the lower line of each prediction band;
determining a range of slopes for a lower upper boundary for the final prediction band based on the distribution of slopes for the lower line of each prediction band;
generating a distribution of intercepts;
determining a range of intercepts based on the distribution of intercepts; and
causing the report to include the range of slopes for the upper boundary, the range of slopes for the lower boundary, and the range of intercepts.

23. The method of claim 22, further comprising:
causing the report to include a graphical representation of the final prediction band based on the range of slopes for the upper boundary, the range of slopes for the lower boundary, and the range of intercepts.

24. The method of claim 13, wherein the genetic sequencing results for each of the multiple libraries is indicative of a number reads corresponding to each oligonucleotide of the plurality of oligonucleotides; and
wherein the method further comprises:
determining, for each of the libraries, a signal value indicative of the number of reads corresponding to an average of the number of reads corresponding to each oligonucleotide of the plurality of oligonucleotides;
calculating a ratio of target concentration for each pair of libraries in the multiple libraries by dividing the higher target concentration of the pair by the lower target concentration of the pair;
calculating a ratio of signal values for each pair of libraries in the multiple libraries by dividing the signal value associated with the library with the higher target concentration of the pair by the signal value associated with the library with the lower target concentration of the pair;
calculating a logarithm of each ratio of target concentration;
calculating a logarithm of each ratio of signal values; and
calculating the prediction band based on a plurality of points each having an x value corresponding to the logarithm of the ratio of target concentration of two libraries and a y value corresponding to the logarithm of the ratio of signal values of the two libraries.

25. A non-transitory computer readable medium containing computer executable instructions that, when executed by a processor, cause the processor to perform a method for determining relative quality of oligonucleotide preparations, the method comprising:
(a) receiving genetic sequencing results for multiple libraries each associated with a target concentration of a plurality of oligonucleotides;
(b) calculating at least one prediction band based on the multiple libraries;
(c) repeating (a) and (b) for a plurality of preparations;
(d) determining boundaries for a final prediction band based on the prediction bands calculated at (b) for each of the plurality of preparations; and
(e) causing to be presented a report indicative of quality of the oligonucleotide libraries associated with the plurality of preparations, wherein the report includes at least metrics indicative of the final prediction band.

26. The non-transitory computer readable medium of claim 25, wherein the method further comprises:
subsequent to (a) and prior to (b), (i) dividing the libraries into a plurality of titer bins based on target concentration, including a high titer bin and a low titer bin; and
repeating (a), (i), and (b) for each of the plurality of preparations.

27. The non-transitory computer readable medium of claim 25, wherein the method further comprises:
receiving genetic sequencing results for multiple new libraries each associated with a target concentration of oligonucleotides;
calculating a prediction band based on the multiple new libraries; and
causing the report to include at least metrics indicative of the prediction band calculated based on the multiple new libraries.

28. The non-transitory computer readable medium of claim 25, wherein the method further comprises:
dividing the new libraries into the plurality of titer bins based on target concentration, including the high titer bin and the low titer bin; and
calculating a prediction band based for each titer band based on the multiple new libraries; and
causing the report to include at least metrics indicative of the prediction band for the high titer bin calculated based on the multiple new libraries.

29. The non-transitory computer readable medium of claim 28, wherein the method further comprises:
causing the report to include a graphical representation of the final prediction band using a first pair of axes; and
causing the report to include a graphical representation of the metrics indicative of the prediction band for the high titer bin calculated based on the multiple new libraries using the same pair of axes.

30. The non-transitory computer readable medium of claim 29, wherein each prediction band includes an upper line and a lower line, wherein the upper line and the lower line are each characterized by a slope m and an intercept c.

31. The non-transitory computer readable medium of claim 31, wherein the method further comprises:
generating a distribution of slopes for the upper line of each prediction band corresponding to the high titer bin;
determining a range of slopes for an upper boundary for the final prediction band based on the distribution of slopes for the upper line of each prediction band corresponding to the high titer bin;
generating a distribution of slopes for the lower line of each prediction band corresponding to the high titer bin;
determining a range of slopes for a lower upper boundary for the final prediction band based on the distribution of slopes for the lower line of each prediction band corresponding to the high titer bin;
generating a distribution of intercepts for the high titer bin;
determining a range of intercepts based on the distribution of intercepts for the high titer bin; and
causing the report to include the range of slopes for the upper boundary, the range of slopes for the lower boundary, and the range of intercepts.

32. The non-transitory computer readable medium of claim 25, wherein the method further comprises:
causing the report to include a graphical representation of the final prediction band using a first pair of axes; and
causing the report to include a graphical representation of the metrics indicative of the prediction band calculated based on the multiple new libraries using the same pair of axes.

33. The non-transitory computer readable medium of claim 25, wherein each prediction band includes an upper line and a lower line, wherein the upper line and the lower line are each characterized by a slope m and an intercept c.

34. The non-transitory computer readable medium of claim 33, wherein the method further comprises:
generating a distribution of slopes for the upper line of each prediction band;
determining a range of slopes for an upper boundary for the final prediction band based on the distribution of slopes for the upper line of each prediction band;
generating a distribution of slopes for the lower line of each prediction band;
determining a range of slopes for a lower upper boundary for the final prediction band based on the distribution of slopes for the lower line of each prediction band;
generating a distribution of intercepts;
determining a range of intercepts based on the distribution of intercepts; and
causing the report to include the range of slopes for the upper boundary, the range of slopes for the lower boundary, and the range of intercepts.

35. The non-transitory computer readable medium of claim 34, wherein the method further comprises:
causing the report to include a graphical representation of the final prediction band based on the range of slopes for the upper boundary, the range of slopes for the lower boundary, and the range of intercepts.

36. The non-transitory computer readable medium of claim 25, wherein the genetic sequencing results for each of the multiple libraries is indicative of a number reads corresponding to each oligonucleotide of the plurality of oligonucleotides; and
wherein the method further comprises:
determining, for each of the libraries, a signal value indicative of the number of reads corresponding to an average of the number of reads corresponding to each oligonucleotide of the plurality of oligonucleotides;
calculating a ratio of target concentration for each pair of libraries in the multiple libraries by dividing the higher target concentration of the pair by the lower target concentration of the pair;
calculating a ratio of signal values for each pair of libraries in the multiple libraries by dividing the signal value associated with the library with the higher target concentration of the pair by the signal value associated with the library with the lower target concentration of the pair;
calculating a logarithm of each ratio of target concentration;
calculating a logarithm of each ratio of signal values; and
calculating the prediction band based on a plurality of points each having an x value corresponding to the logarithm of the ratio of target concentration of two libraries and a y value corresponding to the logarithm of the ratio of signal values of the two libraries.
